(19) United States
(12) Patent Application Publication
(10) Pub. No.: US 2010/0125235 A1
(43) Pub. Date: May 20, 2010

(54) Blood Treatment Apparatus Having Branched Flow Distribution

(75) Inventors: Thomas H. Cauley, III, Berkeley, CA (US); Peter G. Linde, San Ramon, CA (US); Gregory G. Raleigh, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Triaxis Medical Devices, Inc., Foster City, CA (US)

(21) Appl. No.: 12/484,381

(22) Filed: Jun. 15, 2009

Related U.S. Application Data
(60) Provisional application No. 61/061,679, filed on Jun. 16, 2008.

(51) Int. Cl. A61M 1/14

(52) U.S. Cl. 604/5.04

(57) ABSTRACT

Blood treatment apparatus comprise an enclosure having one or more blood treatment modules, typically, hollow fiber bundles, therein. A blood flow inlet distribution network divides an inlet blood flow into a series of successive individual blood flow stages in order to increase uniformity of the distribution of the blood flow into the hollow fiber bundles. The inlet blood flow may be divided in two, three, four, or more stages, providing a large number of end stage flow channels to feed blood into the hollow fiber bundle.

### CROSS-REFERENCES TO RELATED APPLICATIONS

The present application claims the benefit of prior provisional application nos. 61/061,679 (Attorney Docket No. 027543-000100US), filed on Jun. 16, 2008, and 61/172,664 (Attorney Docket No. 027543-000200US), filed on Apr. 24, 2009, the full disclosures of which are incorporated herein by reference.

### BACKGROUND OF THE INVENTION

1. Field of the Invention

The present invention relates generally to medical systems and methods. More particularly, the present invention relates to methods and systems for blood treatment where an inlet flow is divided and distributed to one or more blood treatment modules, such as, hollow fiber bundles.

Patients with kidney failure rely on hemodialysis, hemofiltration, and hemodiafiltration for the removal of toxins and other waste products from their blood. In each of these processes, the blood is exposed to a semipermeable membrane having a molecular weight cut off selected to allow the waste materials to pass through while retaining critical blood proteins. In particular, in hemodialyzers, the blood passes through a bundle of hollow, semipermeable microfibers (typically referred to as a hollow fiber bundle) where the blood flows through the fiber lumens and a dialysis medium (dialysate) flows externally to the fibers. The toxins and other wastes pass from the blood through the semipermeable microfibers into the dialysate being driven by the concentration gradient.

The toxin clearance efficiency of a hemodialyzer is important both to the patient and the treatment facility. To achieve a targeted level of blood cleaning (typically referred to as the urea reduction ratio or URR), there are three variables which must be taken into account. The first is the blood removal rate from the patient. The second is the treatment time. The third is the toxin removal efficiency of the hemodialyzer. It is always advantageous to reduce the treatment time. Not only is it preferable for the patient, shorter treatment times per patient allow the treatment facility to serve the needs of more patients. While the treatment time per patient could theoretically be reduced by increasing the blood removal rate from each patient, the maximum blood flow removal rate is typically determined by patient conditions, such as the patency of their arterio-venous fistula (AVF) or other blood access concerns. Thus, the only practical way to effectively reduce patient treatment times is by improving the toxin removal efficiency of the hemodialyzer.

Blood dialyzer efficiency depends on a number of factors. Of particular interest to the present invention, toxin removal efficiency of the dialyzer depends on the ability to evenly distribute blood flow among all of the individual microfibers in the microfiber bundle. Hemodialysis microfiber bundles typically contain from 1,000 to 500,000 individual microfibers, each of which is subject to blockage and fouling, particularly from coagulation and clotting of blood within the fiber lumen. Such coagulation and clotting can come from slow or stagnant flow within the fiber lumen and/or may originate upstream of the fibers themselves in the flow distribution manifolds and headers of the hemodialyzer. Regardless of the origin of the clotting or coagulation, lost or reduced flow through individual fibers decreases the membrane area available for hemodialysis and causes higher flow rates through the membranes which remain patent. Both these circumstances result in reduced toxin removal efficiencies for the hemodialyzer as a whole.

Other extracorporeal blood treatment systems have complications which are caused or exacerbated by blood flow distribution including bleeding, thromboembolism and heparin induced thrombocytopenia (HIT). Bleeding, which can result from continuous hepain infusion and platelet dysfunction, can be exacerbated by blood flow in the distribution manifold which can cause contact and sheer stress associated activation. Thromboembolism due to thrombus formation within the extracorporeal circuit is a complication which, although infrequent, can be devastating. Heparin-induced thrombocytopenia (HIT), a condition wherein heparin induces immune destruction of platelets, is increasingly common among patients receiving extra corporeal membrane oxygenation (ECMO). When HIT is suspected, the heparin infusion has to be replaced by a non heparin anticoagulant.

Extra corporeal membrane oxygenation (ECMO) is a form of mechanical cardiopulmonary support which is commonly applied intraoperatively to facilitate cardiac surgery which employs the cardiopulmonary bypass technique. Although far less common, prolonged cardiopulmonary support delivered in an intensive care unit is also possible. There are two types of ECMO—venoarterial (VA) and venovenous (VV). Both provide respiratory support, but only VA ECMO provides hemodynamic support. ECMO may be operated in a pump driven circuit or in a pumpless circuit.

ECMO devices are typically constructed of a bundle of gas permeable hollow fibers in a housing where gas passes through the hollow fibers and blood flows around the outside of the fibers. The distribution of blood from is generally accomplished using a single distribution chamber on one side of the hollow fiber bundle and a collection chamber on the exit side. This technology can be applied to ECMO devices where the blood passes either through the hollow fibers or outside the hollow fibers as well as in multiple bundle geometries (round, annular, rectangular).

Pumpless Extracorporeal Lung Assist (PECLA) is a gas exchange device proposed for use in patients with severe gas-exchange disorders such as acute respiratory distress syndrome (ARDS), severe pneumonia and other conditions associated with hypercapnia and hypoxia. PECLA devices are typically constructed of a bundle of gas permeable hollow fibers in a housing where gas passes through the hollow fibers and blood flows around the outside. The distribution of the blood in these devices is accomplished using a single distribution chamber. The high pressure drop across the device limits the flow rate of blood through the device and, thus, the rate of oxygen mass transfer is limited. In addition, the requirement of substantial anticoagulation is necessary to prevent fouling of the device.

Apheresis is the process of removing a specific component from blood and returning the remaining components to a blood donor or patient, in order to collect more of one particular part of the blood than could be separated from a unit of whole blood. One form of apheresis, plasmapheresis is a process usually carried out using a hemofiltration device wherein plasma is separated from blood, and sent to waste. In its place, components such as saline or fresh frozen plasma (FFP) are added; this process has the end result of reducing components in plasma which are harmful to the patient. Examples of diseases commonly treated in this fashion are Goodpasture's syndrome, acute and chronic inflammatory demyelinating polyradiculoneuropathy, Myasthenia gravis and Thrombotic Thrombocytopenia Purpura (TTP).

Hemoconcentrators are devices that perform selective removal of plasma water and some dissolved solutes by way of an ultrafiltration membrane. Hemoconcentrators are routinely used during pediatric cardiopulmonary bypass (CPB) to remove free plasma water and various inflammatory mediators. They employ blood distribution manifolds similar to that of standard dialyzers, and hence require anticoagulation for use, and suffer many of the same complications seen in other blood treatment devices.

For these reasons, it would be desirable to improve the blood flow dynamics through hemodialyzers and other blood treatment apparatus so that uniform and constant flow rates through the individual fibers are maintained over time. In particular, it would be desirable to control the flow dynamics between the blood inlet to the hemodialyzer and the inlet surface of the hollow fiber bundle so that coagulation and clotting are reduced in the inlet manifolds as well as in the individual microfibers.

The use of blood flow distribution vanes and disks has been proposed for improving blood inlet flow dynamics in hemodialyzers. See, for example, DE3 435 883A1; US 2003/0075498; and US 2006/0243653. While such measures might provide some improvement in the blood inlet flow dynamics, the large diameter, low thickness disks which have been proposed fail to address several significant design flaws in present hemodialyzers. For example, they fail to reduce low flow or dead flow volumes within the dialyzers, particularly in the inlet headers or manifolds. Second, the sharp edges of the thin disks and rapid changes in blood flow direction they cause can impart significant shear forces on the blood, which can cause hemolysis, which can cause sheer activation of platelets and result in coagulation in the microfibers of the dialyzer. Additionally, the use of the distribution disks does nothing to relieve the dead flow zones which are found at the interface between the blood inlet manifold or header and the inlet end of the hollow fiber bundle. The individual microfibers are typically spaced apart by a distance several or more times their diameters so that the lumen coverage of the inlet end of the fiber bundle is typically from 15% to 40%, causing regions of both turbulence and dead flow as the blood flow transitions from the inlet manifold into the individual fibers. Such flow risks both have shear forces which can damage the blood as well as blood coagulation and clotting which can occlude the individual fibers.

Thus, there continues to be a need for improved hemodialyzer designs which reduce the risk of coagulation and clotting in fibers and other flow passages.

2. Description of the Background Art

U.S. Pat. No. 7,264,716 describes a filter manifold which divides an inlet flow into four streams which are delivered to four removable and releasable hollow fiber bundles. Blood dialyzers having distribution disks at their inlet ends are described in DE 3435883A1, US 2003/0075498 and US 2006/0243653. U.S. Pat. No. 4,141,835 illustrates a blood dialyzer having a conical flow guide which directs blood to a tubular microfiber bundle. U.S. Pat. No. 6,214,226; U.S. Pat. No. 4,279,542; and U.S. Pat. No. 4,038,191 describe hemodialyzers and other filters having manifolds which divide flows into multiple streams. Blood dialyzers with microfiber bundles having varying densities along their lengths are described in US 2008/0237127; US 2007/0007193; and US 2006/0243653. Other U.S. patents and publications of interest include U.S. Pat. Nos. 7,332,330; 7,264,726; 6,641,731; 6,623,638; 6,426,002; 6,409,024; 6,214,226; 5,139,741; 4,283,284; 4,282,099; 4,261,829; 4,278,542; 4,237,013; 4,211,597; 4,038,191; and 3,746,175.

### BRIEF SUMMARY OF THE INVENTION

The present invention provides blood treatment apparatus designs having a reduced risk of coagulation and clotting in microfibers and other components of the blood flow path. Coagulation and clotting risk is reduced by controlling and maintaining the flow and shear rates through the individual fibers as well as other components of the flow path above a minimum threshold level to avoid stagnation and dead zones which allow coagulation and clotting. The flow rates, however, are limited below a maximum threshold in order to avoid shearing and turbulence which can cause hemolysis, platelet activation (coagulation) and potential degradation of the blood. To achieve these two competing objectives, the hemodialyzers and other blood treatment apparatus are designed to have a very uniform flow velocity distribution over an inlet surface of a blood treatment module, such as among the individual hollow microfibers as well as through other components of the flow path.

Implementation of the present invention to distribute blood to controlled shear and reduce clotting tendency will find it's greatest use in hemodialyzers as described in detail below, but will also find use in a variety of other blood treatment apparutus where blood is distributed over an inlet surface to a blood treatment module. The improved flood inlet flow distribution can have numerous benefits such as a reduced requirement for heparinization. Such a reduction would lead to safer, more efficacious care for patients undergoing ECMO and possibly other treatments. The blood flow distribution technology of the present invention would be beneficial to PECLA technology by reducing the pressure drop by making possible the use of increased numbers of fibers as well as controlled shear levels in blood to mitigate the use of anticoagulation. Thus, the branching flow distribution concepts presented here for hemodialysis can be applied to a number of other extracorporeal blood flitration technologies. Pump driven and pumpless Extracorporeal Membrane Oxygenation (ECMO), Pumpless Extracorporeal Lung Assist (PECLA), Apheresis (plasmapheresis) and Hemoconcentration are a few examples of extracorporeal treatment devices that would benefit from the enhanced flow distribution, reduced anticoagulation requirement and reduced pressure drops achieved with the use of this technology.

Such flow control and uniformity may be achieved by distributing an inlet flow of the blood evenly and uniformly across the inlet surface(s) of one or more hollow fiber bundles being employed in the blood dialyzer. In particular, the present invention provides for dividing or “branching” the inlet blood flow into a plurality of independent, separate flow streams which in turn are distributed evenly and uniformly over the inlet surface(s) of the hollow fiber bundle(s). More particularly, the present invention will provide for at least two stages of such flow division, usually at least three stages of flow division, and often four or more stages of flow division. Usually, the cross-sectional or “flow” areas of each flow channel or path of each successive stage will be reduced to maintain a generally constant flow velocity to further reduce turbulence and flow disruption. Typically, but not necessarily, the total flow in each successive stage will remain substantially constant, with the cross-sectional area of the individual flow path or channel in the successive stage being a fraction of the cross-sectional area of the flow path in the prior stage. That is, if a flow channel at one stage has a total cross-sectional area A, and the flow is divided into “n” flow paths in the next flow stage, the area of each flow path in the successive stage will usually be A/n (assuming that the successive flow paths are intended to carry substantially equal flow volumes).

The use of such flow division or branching allows the inlet blood flow to be divided into a relatively large number of end stage flow channels (i.e. the final flow channels in the flow distribution network which deliver blood to the hollow fiber bundle). Usually, the inlet flow will be divided into at least about 10 end stage flow channels, more usually at least about 25 end stage flow channels, and often about 50 end stage flow channels, and frequently 100 end stage flow channels or more.

Optionally, the flow uniformity from each of the end stage flow channels may be enhanced by providing flow diverter structures which further minimize flow disruption as the blood enters the lumens of the individual microfibers in the fiber bundle. The specific construction and implementation of such flow diverter structures are described in copending application 61/172,664 (Attorney Docket No. 027543-000200US), filed on Apr. 24, 2009, the full disclosure of which is incorporated herein by reference. Such flow diverter structures will typically be provided at the outlet of each individual end stage flow channel between said flow channel and a sector of the hemodialysis microfiber bundle.

The flow distribution networks of the present invention may be employed to deliver uniform blood flow to hollow fiber bundles having a single inlet surface where the plurality of end stage flow channels are uniformly distributed over the single inlet surface. Alternatively, the flow distribution networks may be utilized to deliver flow to the inlet surfaces of a plurality of individual hollow fiber bundles, where one or more such end stage flow channels can provide flow to each of the individual hollow fiber bundles.

Thus, in a first specific aspect of the present invention, a hemodialysis apparatus comprises an enclosure having a blood inlet, a blood outlet, a dialysate inlet, and a dialysate outlet. At least one hollow fiber bundle is disposed within the enclosure and includes an inlet end and an outlet end. The hollow fiber bundle is arranged within the enclosure so that the inlet end receives blood from the blood inlet, and the outlet end delivers blood to the blood outlet. In particular accordance with the present invention, a blood flow distribution network is disposed between the blood inlet of the enclosure and the inlet end of the hollow fiber bundle(s). The blood flow distribution network has at least one passage which receives blood from the blood inlet. Said at least one passage branches into a first plurality of first stage flow channels to achieve a first stage of blood flow distribution. At least some of the first stage flow channels branch into second pluralities of second stage flow channels to achieve a second stage of blood flow distribution. At each stage, the blood flow will be divided into at least two downstream components, usually at least three downstream components, and often four or more downstream components. Typically, each flow channel in each stage will branch into flow channels in the next stage until a sufficient number of branching stages have been achieved, usually at least three stages of branching, often at least four stages of branching, and sometimes more. The last stage of branched flow channels will be referred to hereinafter and in the claims as the “end stage flow channels.” Such end stage flow channels will not further branch and will release blood to the inlet end of the hollow fiber bundle or optionally to a flow diverter structure which in turn releases the blood to the hollow fiber bundle.

In a preferred aspect of the present invention, each stage of branched flow channels will comprise channels having smaller cross-sectional areas than those of the prior stage. That is, the first passage of the blood flow distribution network which receives blood from the blood inlet will have an area adequate to receive the entire volume and flow rate of blood to be treated, typically in the range from 1 mm² to 100 mm². The first passage will then divide into a plurality of first stage flow channels where each of the first stage flow channels has an area which is a fraction of the area of the first passage. That is, if the first passage branches or divides into four first stage flow channels, each of the first stage flow channels will have an area less than that of the first passage, typically being about one-quarter of the cross-sectional area of the first flow passage so that the flow will generally divide equally with minimum change in flow velocity and reduced turbulence. Similarly, the second stage flow channels will have a cross-sectional area which is a fraction of the cross-sectional areas of the first stage flow channels, usually being “1/n,” where n equals the number of branches in the second stage of the flow channels. Such reduction in the cross-sectional area of each successive flow channel stage can be readily determined, and the end stage flow channels will typically have very small cross-sectional areas, usually being in the range from about 0.4 mm² to 4 mm², often in the range from about 0.4 mm² to 2 mm².

In the embodiments where the array of end stage flow channels feeds a single hollow fiber bundle, the end stage flow channels will usually be distributed in a generally uniform pattern over a single, continuous inlet end of the hollow fiber bundle. By “generally uniform,” it is meant that the density of the end stage flow channels over any given unit area will vary by no more than ±25% from the average density, preferably varying by 15% or less.

In the embodiments which employ a multiplicity of individual hollow fiber bundles, the inlet ends of each such hollow fiber bundle will typically be fed by a plurality of the end stage flow channels. Typically, each of the multiplicity of hollow fiber bundles will have the same size and number of hollow fibers, and each inlet end will receive its proportionate share of the total number of end stage flow channels. The end stage flow channels for each of the individual hollow fiber bundles will be uniformly distributed, as defined above, over the inlet ends of the hollow fiber bundle and flow diverters may optionally be used for achieving even greater flow uniformity into the individual hollow fiber bundles. The blood flow distribution networks of the present invention may take a variety of specific forms. The branched flow channels in each stage may be arranged in a plane which is generally parallel to the inlet flow axis. Alternatively, the branching flow channel network may be arranged in one or more planes which is/are generally perpendicular to the inlet blood flow. In other cases, combinations of parallel, perpendicular, and other flow channel orientations may be employed.

In a specific example of a blood flow distribution network in accordance with the present invention, the first and second stage flow channels are arranged in a plane which is generally perpendicular to the axis of blood flow inlet. If a single such planar array is utilized, the end stage flow channels will typically be arranged in a linear array. Optionally, by employing two or more planar distribution arrays in series, a two-dimensional array of end stage flow channels may be provided.

In an alternative embodiment, all stages of the branching flow channels may be arranged in a planar structure or array which may be arranged in parallel with the inlet end(s) of the hollow fiber bundle(s). Optionally, a diffuser plate may be disposed between the perpendicular blood diffuser array and the inlet end of the hollow fiber bundle. The diffuser plate will include a plurality of flow diverters which are arranged to receive blood flow from individual end stage flow channels and to deliver the blood to a sector of the individual hollow fibers in the bundle.

In a further aspect of the present invention, methods for dialyzing a blood flow comprise dividing the blood flow into a first plurality of first stage flows. At least some of the first stage flows are divided into a second plurality of second stage flows, and optionally at least some of the second stage flows may be divided into a third plurality of third stage flows and at least some of the third stage flows may be divided into fourth stage flows. While four such flow divisions will usually be sufficient, it will be appreciated that fifth, sixth, and even more stages of flow division may be provided within the scope of the present invention. The final divided flow stage will be referred to as the “end stage flow,” and each of the end stage flows are delivered to an inlet end of a hollow fiber bundle, and the blood flow through the hollow fiber bundle is dialyzed using a dialysate flow in an otherwise conventional manner.

The end stage flows may be delivered to an inlet end of the single hollow fiber bundle, generally as described above with respect to the apparatus of the present invention, or the end stage flows may be separately delivered to a plurality of inlet ends of a plurality of hollow fiber bundles. In all cases, the end stage flows will preferably be substantially uniformly distributed over the inlet end(s) of the hollow fiber bundle(s), where uniformity has been previously defined.

The initial blood flow to be dialyzed is usually oriented in an axial direction, and the blood flow may be divided into successive stage flows in one or more planar arrays of flow channels. For example, the blood flow may be divided in a single planar array having two, three, four, or more stages of flow division, where the end stage flow channels will typically be arranged in a linear array. Alternatively, the inlet blood flow may pass through successive planar arrays of branching flow channels where a first array may provide a linear array of flow channels and a plurality of secondary planar arrays may receive blood from each of the flow channels of the first planar array to further divide those flows into multiple end stage or intermediate stage flows. Using successive plane arrays can provide for two-dimensional end stage flow channel arrays.

Alternatively, the inlet blood flow may be initially diverted to flow in a planar array which is perpendicular to the blood flow and parallel to the inlet end(s) of the hollow fiber bundle(s). In such instances, the perpendicular flow channel arrays may provide for a highly uniform two-dimensional array of end stage flow channels for uniformly delivering blood to the inlet end(s) of the hollow fiber bundle(s). Optionally, flow diverter structures may be provided between the two-dimensional end stage arrays and the inlet end(s) of the hollow fiber bundle(s).

### BRIEF DESCRIPTION OF THE DRAWINGS

FIG. 1 is a schematic illustration of a flow distribution network constructed in accordance with the principles of the present invention delivering blood to an inlet end of a single hollow fiber bundle.

FIG. 2 is a schematic view similar to FIG. 1, where the flow distribution network is delivering blood to a plurality of inlet ends of a plurality of hollow fiber bundle(s).

FIGS. 3A and 3B illustrate flow division according to the present invention in three dimensions.

FIG. 4 illustrates the use of planar flow distribution arrays for delivering divided flow to a plurality of individual hollow fiber bundles.

FIG. 5 illustrates an alternative flow distribution network where the divided flows recombine to deliver a plurality of end stage blood flows to the inlet end of a single hollow fiber bundle.

FIG. 6 is similar to FIG. 5, except that the flow distribution network is delivering flow to a plurality of individual hollow fiber bundles.

FIG. 7 is a perspective view of a dialyzer cap constructed in accordance with the principles of the present invention with portions broken away.

FIG. 8 is the top view of the dialyzer cap of FIG. 7, with portions broken away illustrating four stages of flow diversion in a single plane.

FIG. 9 is a bottom view of the dialyzer cap of FIG. 7 illustrating a plurality of diffuser cells which receive blood from the end stage flow channels shown in FIG. 8.

FIG. 10 is a schematic illustration of how the flow in the dialyzer cap of FIG. 7 progresses from the end stage flow channels to the diffuser cells and then to the inlet surface of the hollow fiber bundle of the dialyzer.

### DETAILED DESCRIPTION OF THE INVENTION

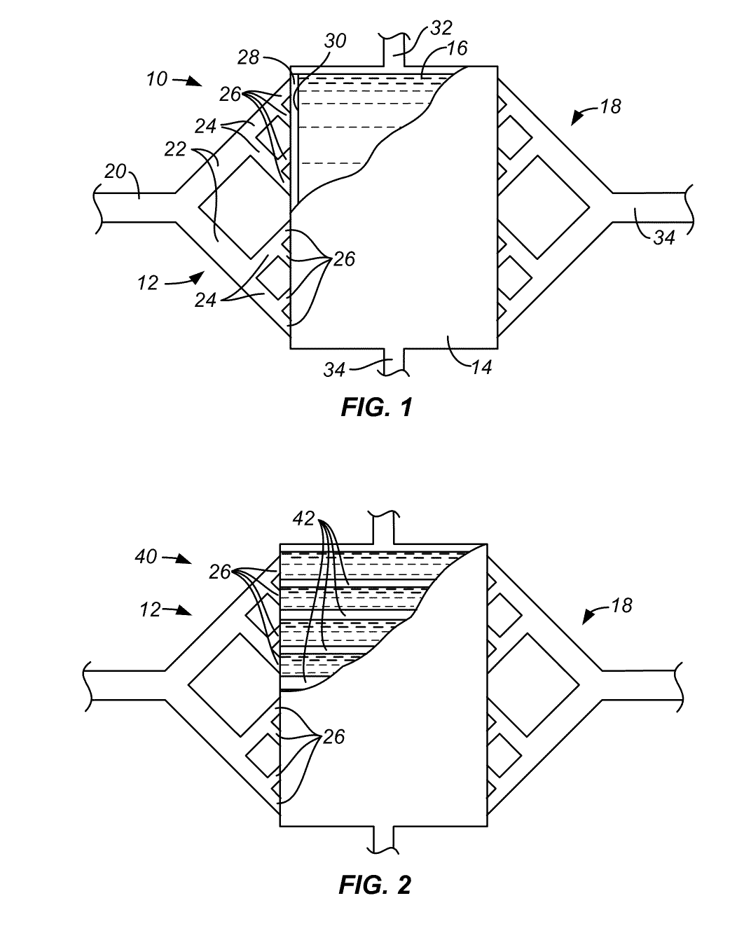

Referring now to FIG. 1, a first exemplary hemodialyzer 10 constructed in accordance with the present invention comprises an inlet flow distribution network 12, an enclosure 14, a hollow fiber bundle 16 within the enclosure, and an outlet flow distribution network 18. A flow of blood to be treated enters in through a main passage 20 which divides into a pair of first stage flow channels 22, each of which in turn are divided into a pair of second stage flow channels 24. The second stage flow channels 24, in turn, each divide into a pair of third stage flow channels 26, thus providing a total of eight end stage flow channels delivering a distributed blood flow to the hollow fiber bundle 16 within the enclosure 14. In the embodiment of FIG. 1, the eight third stage (end stage) flow channels 26 deliver the blood into an open plenum 28 which lies between the outlets of the flow channels 26 and the inlet surface 30 of the hollow fiber bundle 16. The division of the inlet flow through passage 20 into eight generally equal outlet flows through the end stage flow channels 26 will be sufficient to enhance the flow uniformity and dialyzer efficiency. Various flow diverter structures could be provided within the plenum 28, as described in commonly owned, copending application 61/172,664 (Attorney Docket No. 027543-000200US), the full disclosure of which has been previously incorporated herein by reference.

Blood passing through the hollow fiber bundle 16 will be dialyzed by a dialysate flow which can be introduced through dialysate inlet 32 and collected at dialysate outlet 34. As illustrated, the outlet flow collection network 18 is a mirror image of the inlet flow distribution network 12, where eight separate inlet flow channels collect the blood and converge through stages into a single outlet passage 34. While use of such a converging flow collection network is generally preferred, it will be appreciated that flow uniformity is less critical after the blood has passed through the flow dialyzer and a variety of other flow collection manifolds and networks could be implemented in place of the illustrated flow collection network 18.

A second exemplary hemodialyzer 40 is illustrated in FIG. 2. Hemodialyzer 40 is similar in most respects to the first exemplary hemodialyzer 10, including an identical inlet flow distribution network 12 and outlet flow collection network 18. The hemodialyzer 40 differs from dialyzer 10, however in that a plurality of individual hollow fiber bundles 42 are provided, with each bundle being aligned with one end stage flow channel 26 and with the corresponding inlet flow channel in the outlet collection network 18. By generally matching the area of the end stage flow channels 26 with the cross-sectional area of the hollow fiber bundles 42, enhanced flow distribution and reduced flow turbulence can be achieved, thus providing for improved dialysis efficiency. Dialysis efficiency might be further improved by employing hollow fiber bundles 42 having closely packed inlet ends, as described in commonly owned, copending application 61/172,664 (Attorney Docket No. 027543-00011 US), the full disclosure of which has been previously incorporated herein by reference.

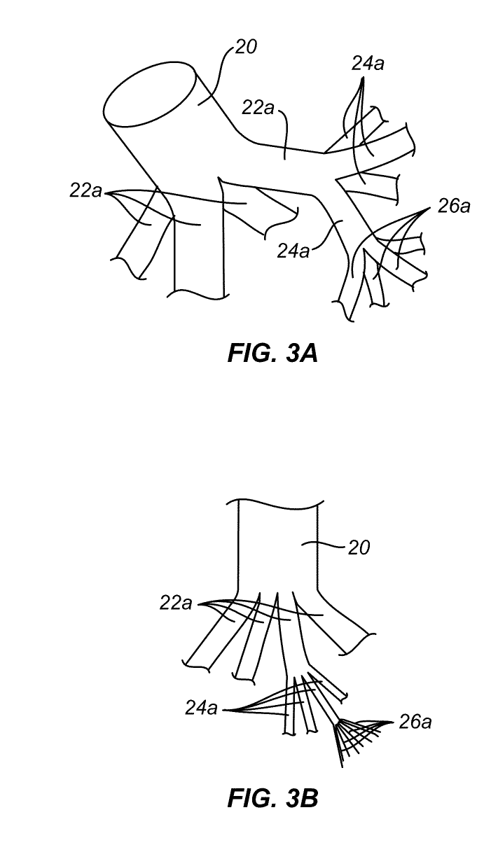

As illustrated in both FIGS. 1 and 2, the flow distribution networks 12 and collection networks 18 are shown to have generally “planar” configurations where the end stage flow channels 26 are arranged in a linear array. While such structures are useful and achieve many of the objectives of the present invention, it will often be desirable to use cylindrical, square, or other hemodialyzer configurations having a two-dimensional inlet end. It will be appreciated, as shown in FIGS. 3A-3B, that the branching flow distribution networks can be implemented in three dimensions. For example, as shown in FIG. 3A, the inlet passage 20 could branch into four radically diverging first stage flow channels 22A, with each of the four first stage flow channels in turn branching into four second stage flow channels 24a, and in turn branching into four third stage flow channels 26a. With just three stages of branching, with four branches at each stage, the single initial flow through passage 20 will be divided into 64 end stage flows through flow channels 26a. Such branching is also illustrated in FIG. 3B where the image is flattened out to show each stage of branching and the general relative size of the flow channels in each stage.

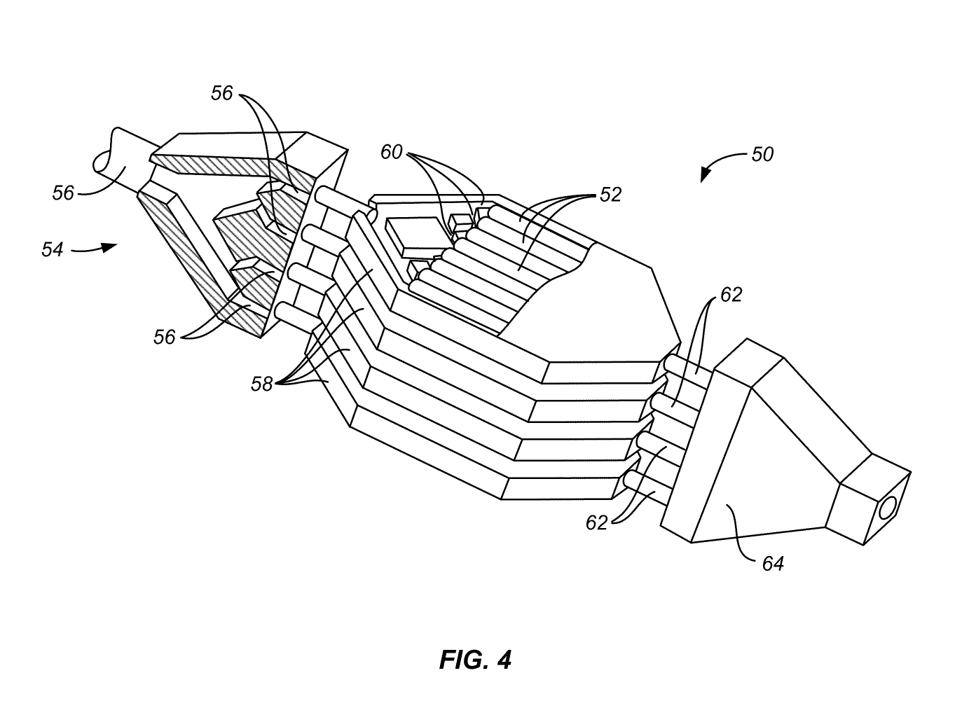

The provision of two-dimensional end stage arrays can be achieved in other ways. For example, as shown in FIG. 4, a plurality of planar arrays can be joined in a series-parallel arrangement to feed a rectangular array of individual hollow fiber bundles 52. The hemodialyzer 50 includes a first planar array 54 which includes an inlet passage 56 which opens into a network of passages which divide the flow through first and second stages into four second stage outlets 56. Each of the second stage flow channels 56, in turn, is connected to one additional planar array 58 which further divides the flow into eight flow channels 60 to feed the eight hollow fiber bundles 52 which are in that planar structure. It will be appreciated that the planar arrays 58 could be constructed similarly to the array 40 shown in FIG. 2. The four planar arrays 58 are vertically stacked to, in effect, provide a two-dimensional array including a total of 32 hollow fiber bundles 54 arranged in a generally square or rectangular configuration. Outlet ends 62 of the four planar arrays 58 are delivered to an outlet collection array 64 which may be constructed similarly to the inlet array 54.

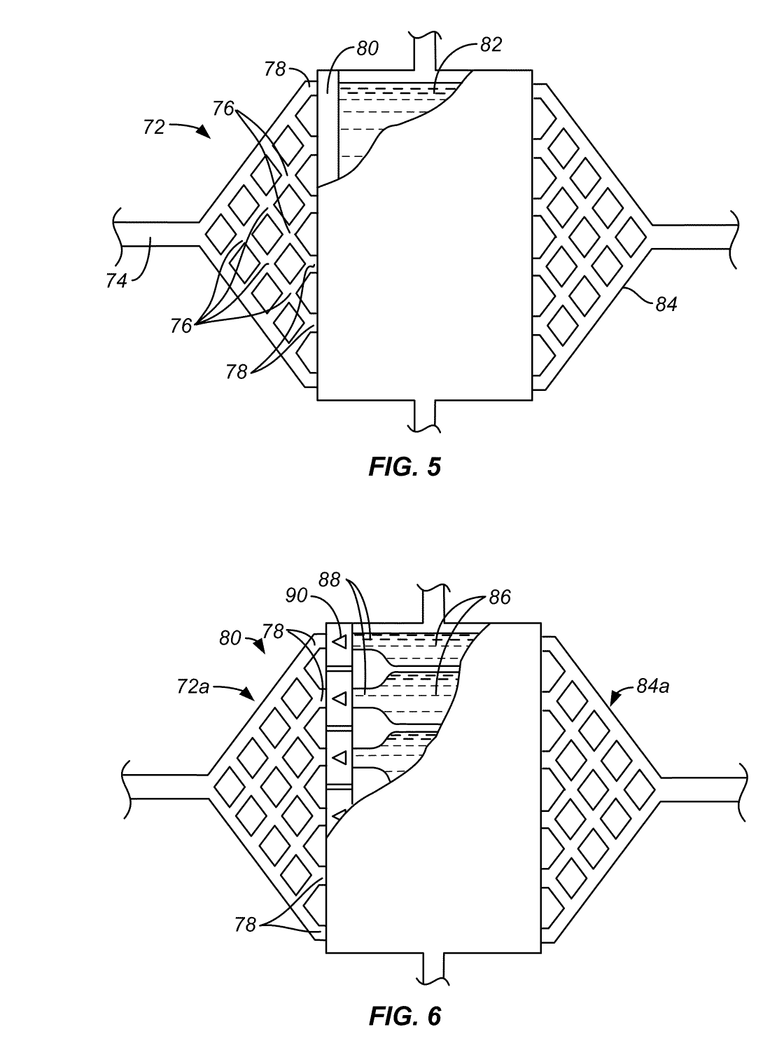

The flow distribution networks described thus far provide for branching of the flow at each node or branch point. In some instances, it may be desirable to both branch and reconnect the flows at successive nodal points, as shown in FIGS. 5 and 6. A fourth exemplary hemodialyzer 70 is illustrated in FIG. 5 and includes an inlet flow distribution network 72 where the inlet passage 74 branches at a number of nodal points 76, eventually providing six end stage flow channels 78. The six flow channels 78 feed blood into plenum 80 which in turn flows the blood into a single hollow fiber bundle 82. Blood from the hollow fiber bundle 82 flows into an outlet distribution network 84 which may be constructed similarly to the inlet distribution network 72.

As shown in FIG. 6, a fifth exemplary hemodialyzer 80 may include inlet and outlet flow distribution networks 72a and 84a which are identical to those shown in FIG. 5. The hemodialyzer, in contrast, employs a plurality of individual hollow fiber bundles 86, where each bundle receives blood from a single one of the end stage flow channels 78. The individual hollow fiber bundles 86 may employ tightly packed inlet ends 88 of the type described in commonly owned, copending application 61/172,664 (Attorney Docket No. 027543-000200US), the full disclosure of which has previously been incorporated herein by reference. The hemodialyzer 70 may further include a plurality of flow diverters 90 to further enhance the blood flow into the constricted inlet ends of the hollow fiber bundles 88.

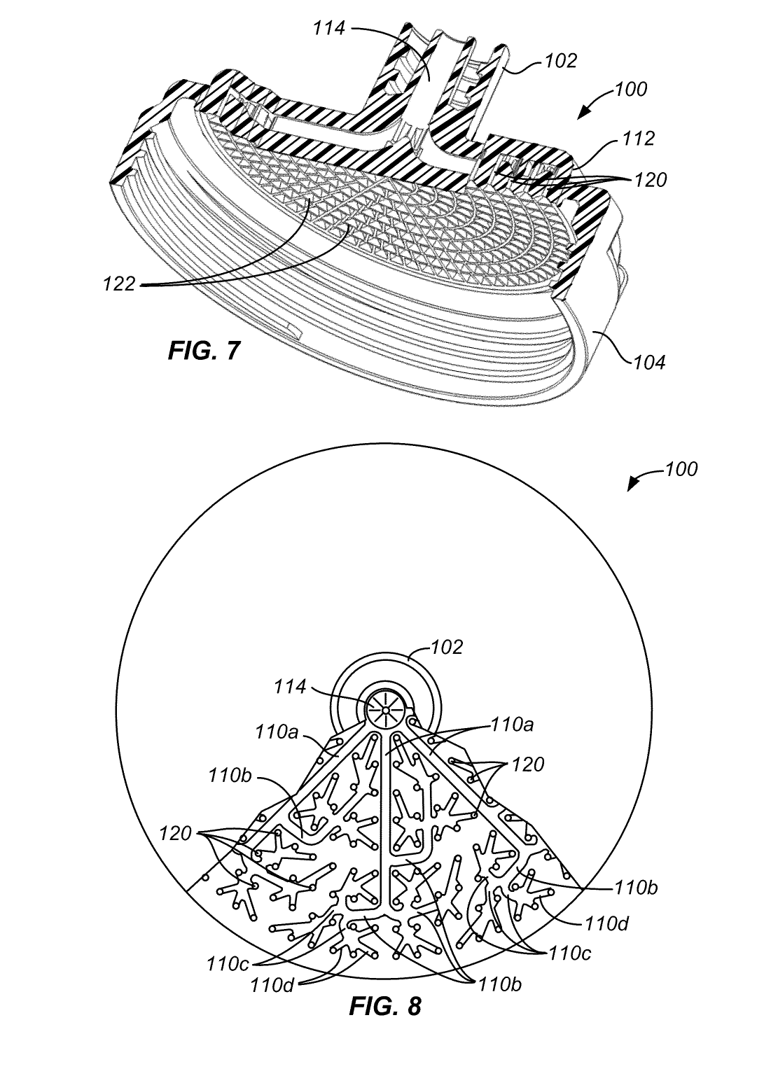

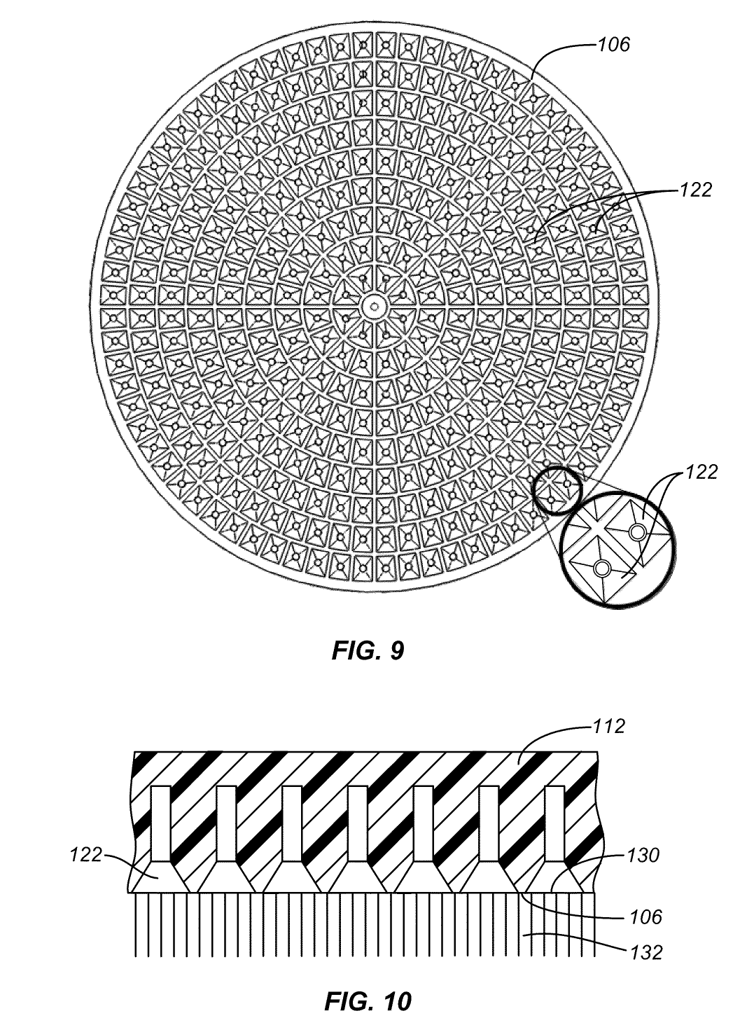

A dialyzer end cap 100 is illustrated in FIGS. 7-9 and is of a type which can be mounted at either of the inlet or outlet end of a conventional hollow fiber dialysis bundle. The dialysis end cap 100 includes an inlet port connector 102 which can be connected to a conventional blood inlet flow line and a sidewall 104 which can be threaded on to the end of the hollow fiber dialysis bundle housing (not shown). Between the blood flow port inlet 102 and a bottom surface 106 of the end cap 100, a plurality of dividing or branching flow channels 110 are formed in a disk portion 112 of the end cap. As best seen in FIG. 8, an axial inlet passage 114 divides into a plurality of first stage flow channels 110a which in turn branch into a plurality of second stage flow channels 110b. The second stage flow channels 110b, in turn, divide into a plurality of third stage flow channels 110c which, in turn, divide into a plurality of fourth stage flow channels 110d, which are the end stage flow channels. In the particular embodiment of FIGS. 7-9, the axial inlet flow channel 110 divides into a total of eight first stage flow channels, which each divide into a total of three second stage flow channels 110b, which each in turn divide into a total of three third stage flow channels 110c, which each divide into five fourth or end stage flow channels 110d. Thus, the single axial flow channel 110 will ultimately divide into a total of 360 end stage flow channels. Each end stage flow channel, in turn, will terminate in a via 120, i.e. a vertical passage through the thickness of the disk portion 112 of the end cap, as best seen in FIG. 7. The vias 120, in turn, each terminate in a diffuser cell 122 formed in the bottom surface 106 of the end cap, as best seen in the enlarged portion of FIG. 9. The diffuser cells 122 (of which the full 360 are illustrated in FIG. 9 to evenly distribute or spread the blood over the inlet end 130 of a hollow fiber bundle 132, as shown schematically in FIG. 10. Optionally, each of the diffuser cells 122 could include a diverter structure (not shown but previously described) in order to further enhance the flow distribution.

While the above is a complete description of the preferred embodiments of the invention, various alternatives, modifications, and equivalents may be used. Therefore, the above description should not be taken as limiting the scope of the invention which is defined by the appended claims.

What is claimed is:

1. Extracorporeal blood treatment apparatus comprising:
an enclosure having a blood inlet and, a blood outlet;
a blood treatment module having an inlet end and an outlet end, wherein the module is arranged in the enclosure so that the inlet end receives blood from the blood inlet and the blood outlet receives blood from the outlet end; and
a blood flow distribution network disposed between the blood inlet of the enclosure and the inlet end of the blood treatment module, said blood flow distribution network having at least one passage which receives blood from the blood inlet, wherein said at least one passage branches into a first plurality of first stage flow channels, wherein at least some of the first stage flow channels branch into second pluralities of second stage flow channels, and wherein end stage flow channels which do not further branch release blood to the inlet end of the hollow fiber bundle.

2. Apparatus as in claim 1, wherein the blood treatment comprises a hollow fiber module and said enclosure has a dialysate inlet and a dialysate outlet.

3. Apparatus as in claim 1, wherein at least some of the channels of the second pluralities of second stage flow channels branch into third pluralities of third stage flow channels.

4. Apparatus as in claim 3, wherein at least some channels of the third pluralities of third stage flow channels branch into fourth pluralities of fourth stage flow channels.

5. Apparatus as in claim 1, wherein the first plurality of first stage flow channels have cross-sectional areas which are less than the cross-sectional areas of the at least one passage.

6. Apparatus as in claim 4, wherein the second plurality of second stage flow channels have cross-sectional areas less than those of the first stage flow channels.

7. Apparatus as in claim 1, wherein the total cross-sectional flow area of each successive stage of flow channels is substantially constant.

8. Apparatus as in claim 2, consisting essentially of a single hollow fiber bundle having a single, continuous inlet end, wherein all end stage flow channels feed blood to said inlet end.

9. Apparatus as in claim 8, wherein said end stage flow channels are distributed in a generally uniform pattern over said single, continuous inlet end of the hollow fiber bundle.

10. Apparatus as in claim 2, comprising a multiplicity of individual hollow fiber bundles each having an inlet end and an outlet end, wherein at least one end stage flow channel feeds each individual hollow fiber bundle.

11. Apparatus as in claim 10, wherein the inlet ends of the individual hollow fiber bundles each have generally equal areas and are fed by the same number of end stage flow channels.

12. Apparatus as in claim 11, wherein the number of end stage flow channels is equal to the number of fibers in the hollow fiber bundle.

13. Apparatus as in claim 2, wherein the blood inlet is axially aligned with an axis of the hollow fiber bundle and wherein the first and second stage flow channels are arranged in a plane which is generally perpendicular to the axis.

14. Apparatus as in claim 13, further comprising a diffusion plate disposed between the blood flow distribution network and the inlet end of the hollow fiber bundle, wherein said diffuser plate includes a plurality of diffuser cells which each receive blood from one end stage flow channel and deliver the blood to a sector of individual hollow fibers in the bundle.

15. Apparatus as in claim 14, wherein at least some of the diffuser cells include diverter structures to distribute blood uniformly within the sector.

16. Apparatus as in claim 2, wherein the blood inlet is axially aligned with an axis of the hollow fiber bundle(s) and wherein the first and second stage flow channels are arranged in at least one planar array which is generally parallel to the axis.

17. Apparatus as in claim 16, wherein the blood flow distribution network consists essentially of a single planar array with the end stage flow channels terminating in a linear array.

18. Apparatus as in claim 16, comprising a multiplicity of individual hollow fiber bundles each having an inlet end and an outlet end, wherein said individual hollow fiber bundles are arranged in a planar configuration with said inlet ends arranged in a linear pattern to receive blood from the linear array of end stage flow channels.

19. Apparatus as in claim 16, wherein successive stages of flow channels are arranged in successive planar arrays, with a final planar array having end stage flow channels terminating in a two dimensional array.

20. Apparatus as in claim 19, wherein the blood flow distribution network consists essentially of a first planar array with “n” flow channels terminating in a linear array and “n” successive planar arrays each of which receives blood from one terminating flow channel in the first planar array.

21. Apparatus as in claim 20, consisting essentially of a single hollow fiber bundle having a single inlet end which is arranged to receive blood from the two dimensional array of end stage flow channels.

22. Apparatus as in claim 20, comprising a multiplicity of individual hollow fiber bundles each having an inlet end and an outlet end, wherein said inlet ends of said individual hollow fiber bundles are arranged in a two-dimensional array such that individual inlet ends receive blood from individual end stage flow channels.

23. A method for dialyzing a blood flow, said method comprising:
dividing the blood flow into a first plurality of first stage flows;
dividing at least some of the first stage flows into a second plurality of second stage flows;
delivering end stage flows to an inlet end of a hollow fiber bundle; and
dialyzing the blood as the blood passes through the hollow fiber bundle.

24. A method as in claim 23, further comprising dividing at least some of the second stage flows into a third plurality of third stage flows.

25. A method as in claim 24, further comprising dividing at least some of the third stage flows into a fourth plurality of fourth stage flows.

26. A method as in claim 23, wherein all end stage flows are directed to a single hollow fiber bundle.

27. A method as in claim 26, wherein the end stage flows are distributed substantially uniformly over an inlet end of the hollow fiber bundle.

28. A method as in claim 27, wherein each end stage flow is directed to a single fiber in the hollow fiber bundle.

29. A method as in claim 23, wherein at least some individual end stage flows are directed to different hollow fiber bundles.

30. A method as in claim 23, wherein the blood flow is oriented in an axial direction and the blood flow is divided into successive stage flows in at least one planar array of flow channels.

31. A method as in claim 30, wherein the blood blow is divided in a single planar array disposed perpendicularly to the axial direction.

32. A method as in claim 31, wherein the single planar array is disposed adjacent to a single inlet end of a single hollow fiber bundle, wherein the end stage flows are distributed substantially uniformly over the single inlet end.

33. A method as in claim 31, wherein the blood flow is divided in two or more planar arrays disposed in parallel to the axial direction.

34. A method as in claim 33, wherein the blood flow is divided into a linear array of divided flows in a first planar array.

35. A method as in claim 34, wherein the blood flow is further divided into a two-dimensional array of divided flows in a plurality of additional planar arrays each of which receives blood from one of the flows in the linear array.
